(19) United States
(12) Patent Application Publication
(10) Pub. No.: US 2011/0256187 A1
(43) Pub. Date: Oct. 20, 2011

(54) BIOFILM-INHIBITORY COATINGS THAT RELEASE SALICYCLIC ACID BY HYDROLISIS

(75) Inventors: Edwin R. Hortelano, Pewaukee, WI (US); Paul J. Nowatzki, Pittsburgh, PA (US)

(73) Assignee: BAYER MATERIALSCIENCE LLC, Pittsburgh, PA (US)

(21) Appl. No.: 13/128,441

(22) PCT Filed: Nov. 13, 2009
(86) PCT No.: PCT/US2009/006106

§ 371 Date: Jun. 27, 2011

Related U.S. Application Data
(60) Provisional application No. 61/115,213, filed on Nov. 17, 2008.

(51) Int. Cl. A61K 9/00; A61P 31/00; C08F 222/22; A61K 31/785; C08F 2/46

(52) U.S. Cl. 424/400; 424/78.35; 427/2.3; 522/174; 526/301

(57) ABSTRACT

The present invention relates to coating compositions for modulating biofilm growth on a surface, the composition comprising the reaction product of:
A) an ethylenically unsaturated compound, having at least one group with the following structure:

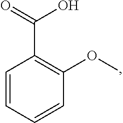

and
B) an ethylenically unsaturated compound.

### BACKGROUND OF THE INVENTION

Catheter-associated urinary tract infections (CAUTIs) have proven; frustratingly difficult to prevent or even reduce in frequency. Catheters that release biocidal silver in order to combat CAUTIs have been successful in the marketplace, but evidence on their clinical effectiveness is mixed. While antibiotics have proven successful in treating the free-floating, planktonic bacteria that cause systemic infections, they often fail against device-associated infections because on non-biologic surfaces, bacteria form persistent biofilms. The resistance of biofilms to antibiotics, at up to 1000 times the concentrations required to kill corresponding planktonic bacteria, is attributed in part to their complex architectures and differentiation of function.

Several distinct microorganisms have been observed to cause CAUTIs, including Gram positive and Gram negative bacteria as well as yeast. Thus, to combat biofilms and biofilm-associated infections, there is a strong preference for treatments with a broad spectrum of activity. Once formed, biofilms are very hard to remove, so most research into anti-infective medical devices aims to devise materials that kill microorganisms as soon as they touch or come near a surface.

Another possibility for preventing biofilms and associated infections, which has had limited commercial impact to date, is to disrupt or inhibit the formation of biofilms without necessarily killing the microorganism. Approaches of this type are termed anti-biofilm. For instance, chemicals are known that interfere with bacterial quorum sensing systems, the external signals bacteria use to modify their gene expression. If biofilm formation can be inhibited, bacterial cell density on the surface can be limited and it is expected that the virulence of a bacterial colony on a device could be attenuated. This area of research has recently attracted considerable attention, and a number of candidate molecules have been suggested as potent quorum sensing inhibitors.

Salicylic acid (SA), a metabolite of acetylsalicylic acid (aspirin), has been noted to have a wide variety of effects on many species bacteria, including the disruption of biofilm formation and the attenuation of virulence factors. While the mechanisms are not completely understood, salicylic acid has been demonstrated to inhibit biofilms of Staphylococcus epidermidis, Bacillus subtilis, and Pseudomonas aeruginosa; and Staphylococcus aureus, to reduce the synthesis of virulence factors in Pseudomonas aeruginosa and Staphylococcus aureus, and to reduce adhesion of various organisms to catheters. SA has anti-biofilm effects on both Gram positive (Staphylococcus) and Gram negative (Pseudonionas) bacteria, as does aspirin against the yeast Candida albicans.

Salicyl acrylate and polymers thereof are known, with imparting anti-inflammatory properties to a polymer being an early goal of such materials; hemocompatibility is another mentioned target for similar materials.

WO 0027438 and US 2002/016278 disclose the use of 0.2% sodium salicylate in mixed detergent/antibacterial formulations for removing biofilms, and U.S. Pat. No. 6,585,961 discloses salicylic acid as an optional additive to antimicrobial compositions.

US 2003/153983 and WO 2005-20050224 disclose implantable medical devices comprising biocompatible polymers that include both a biocide and an anti-adhesion or anti-bacterial agent, and teach polyurethane catheters including triclosan and salicylic acid.

However, none of the mentioned publications teach a material that is capable of releasing salicylic acid for the purpose of inhibiting biofilm formation or growth, while at the same time, maintaining its structural integrity. Thus, an object of the present invention is to provide such a material, which will release salicylic acid by hydrolysis, and will remain structurally intact after the release.

### SUMMARY OF THE INVENTION

The present invention is directed to a coating composition comprising the reaction product of:
A) an ethylenically unsaturated compound having at least one group with the following structure:

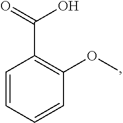

and
B) an ethylenically unsaturated compound having at least one urethane group.

The present invention is also directed to a method for modulating biofilm growth on a surface, comprising coating said surface with a composition comprising the reaction product of:
A) an ethylenically unsaturated compound having at least one group with the following structure:

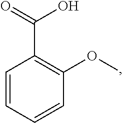

and
B) an ethylenically unsaturated compound.

The present invention is also directed to a method of producing a polyurethane material comprising

1) mixing:
A) an ethylenically unsaturated compound having at least one group with the following structure:

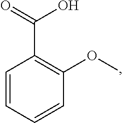

and
B) an ethylenically unsaturated compound having at least one urethane group; and

2) exposing the mixture to actinic radiation.

### DETAILED DESCRIPTION OF THE INVENTION

The present invention is directed to a coating composition comprising the reaction product of:
A) an ethylenically unsaturated compound having at least one group with the following structure:

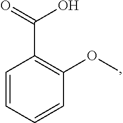

and
B) an ethylenically unsaturated compound having at least one urethane group.

Component A) may include any ethylenically unsaturated compounds that include a salicyl acid group. Examples include, but are not limited to, salicyl acrylate, salicyl methacrylate and bis(salicyl) fumarate.

Compounds suitable for use as component A) may be produced by the reaction of salicylic acid and unsaturated acid chlorides in the presence of a base, or by reaction of salicylate salts and unsaturated acid chlorides. Examples of suitable unsaturated acid chlorides include, but are not limited to, acryloyl chloride, methacryloyl chloride, fumaryl chloride, and itaconyl chloride. Examples of bases include a wide range of bases, including but not limited to, triethylamine or pyridine. Examples of salicylicate salts include, but are not limited to, sodium salicylate, potassium salicylate, lithium salicylate, and organic salicylate salts.

Compounds suitable for use as component A) could, be made by the reaction of anhydrides, with salicylic acid or salicylate. Examples of suitable anhydrides include, but are not limited to, acrylic anhydride, methacrylic anhydride, crotonic anhydride, and tiglic anhydride.

Component B) may include any ethylenically unsaturated compound having at least one urethane group. In this regard, preferred urethane acrylates are also described in U.S. Pat. Nos. 4,380,604, 6,232,360, 6,753,394 and 6,790,485. Such urethane (meth)acrylates are generally prepared by reacting one more polyisocyanates with one or more hydroxyl group-containing unsaturated (meth)acrylates, and optionally with additional hydroxyl group-containing molecules.

Suitable polyisocyanates include organic polyisocyanates having aliphatically, cycloaliphatically and/or aromatically bound isocyanate groups and generally having molecular weights of from about 144 to about 1000, more preferably from about 168 to about 300. Suitable examples include butylene diisocyanate, hexamethylene diisocyanate isophorone diisocyanate (IPDI), 3(4)-isocyanatomethyl-methylcyclohexyl isocyanate (IMCI), trimethylhexamethylene diisocyanate (2,2,4 and/or 2,4,4-trimethyl-hexamethylene diisocyanate), the isomeric bis(4,4′-isocyanato-cyclohexyl)methanes (H₁₂MDI), the isomeric bis(isocyanato-methyl)-methylcyclohexanes, isocyanatomethyl-1,8-octane diisocyanate, 1,4-cyclohexylene diisocyanate, 1,4-phenylene diisocyanate, 2,4- and/or 2,6-toluylene diisocyanate (TDI), 1,5-naphthylene diisocyanate, 2,4′- and/or 4,4′-diphenylmethane diisocyanate (MDI), triphenylmethane-4,4′,4″-triisocyanate or their derivatives having a urethane, isocyanurate, allophanate, biuret, uretdione, iminooxadiazinedione structure and/or mixtures thereof as well as mixtures of aliphatic and aromatic diisocyanates and/or polyisocyanates. The production of such derivatives is known and described, for example, in U.S. Pat. Nos. 3,124,605, 3,183,112, 3,919,218, and 4,324,879 and in European patent 798 299.

Preferably used are HDI, IPDI, TDI, H₁₂MDI and/or isocyanurate group-containing polyisocyanates obtained by trimerization of HDI, TDI or IPDI. Particularly preferred are HDI and IPDI and mixtures thereof.

In preparing the unsaturated urethane (meth)acrylate, the polyisocyanate is reacted at an isocyanate to OH equivalent ratio of from about 0.95:1 to about 1:0.95 (and more preferably about 1:1) with i) an unsaturated polyether (meth)acrylate having an OH number of from about 30 to about 300, ii) a mono-, di-, tri-, or polyhydroxyl C₁ to C₁₀-alkyl or C₆ to C₁₀-aryl (meth)acrylate, or iii) a mixture thereof. The resultant unsaturated urethane (meth)acrylate will have an isocyanate group content of less than 1%, by weight. It is also possible to prepare useful unsaturated urethane (meth)acrylates with residual isocyanate functionality, by reacting the polyisocyanate at an isocyanate-to-OH equivalent ratio of from about 1:1 to 10:1. The resultant isocyanate-functional urethane meth(acrylate) will have an isocyanate group content of greater than 1% by weight, preferably between about 3% and 20% by weight.

Useful unsaturated polyether (meth)acrylates are prepared by reacting a polyether polyol (having an hydroxyl functionality of from 2 to 6) with acrylic and/or methacrylic acid. Suitable polyether polyols are of the type known in the polyurethane art and are generally prepared by reacting a suitable staring molecule (such as, e.g., ethylene glycol, propylene glycol, butanol, glycerol, trimethylol propane, hexane diol, pentaerythritol and the like) with ethylene oxide, propylene oxide or a mixture thereof. The polyether is then reacted with acrylic and/or methacrylic acid. When the unsaturated (meth)acrylate is to be used to prepare the unsaturated urethane (meth)acrylate), the polyether is selected so as to produce the (meth)acrylate having the required OH number and the components are reacted in amounts such that the resultant unsaturated polyether (meth)acrylate has an OH number of from about 30 to about 500, preferably from about 100 to about 400 and most preferably from about 200 to about 300. In the case where the unsaturated (meth)acrylate is to be used as an part or all of component A), the polyether is selected so as to produce the (meth)acrylate having the required OH number and, the polyether and acrylic (and/or methacrylic) acid are reacted in amounts such that the resultant unsaturated polyether (meth)acrylate has an OH number of from about 30 to about 100, preferably from about 100 to about 400 and most preferably from about 200 to about 300.

Useful mono-, di-, tri-, or polyhydroxyl. C₁ to C₁₀-alkyl or C₆ to C₁₀-aryl (meth)acrylates are also known in the polyurethane art. Such material are prepared by reacting relatively low molecular weight diols, triols and polyols (such as ethylene glycol, propylene glycol, butanol, glycerol, trimethylol propane, hexane diol, pentaerythritol and the like) with acrylic and/or methacrylic acid in amounts such that the resultant product contains one or more hydroxyl groups. Specific examples include hydroxyethyl acrylate, hydroxypropyl acrylate, hydroxybutyl acrylate, hydroxypropyl acrylate, hydroxyhexyl acrylate, triglycerol diacrylate, dipentaerythritol pentaacrylate, and the corresponding methacrylates.

The coating composition described in this invention may also comprise one or more initiators that can generate free radicals. An initiator can be a photoinitiator, a thermal initiator, a redox initiator, or another initiator. A photoinitiator can be substantially any photoinitiator. A variety of photoinitiators can be utilized in the radiation-curing compositions of the present invention. The usual photoinitiators are the type that generate free radicals when exposed to radiation energy. Suitable photoinitiators include, for example, aromatic ketone compounds, such as benzophenones, alkylbenzophenones, Michler's ketone, anthrone halogenated benzophenones. Further suitable compounds include, for example, 2,4,6-trimethylbenzoyldiphenylphosphine oxide, phenylglyoxylic acid esters, anthraquinone and the derivatives thereof, benzil ketals and hydroxyalkylphenones. Illustrative of additional suitable photoinitiators include 2,2-diethoxyacetophenone; 2- or 3- or 4-bromoacetophenone; 3- or 4-allyl-acetophenone; 2-acetonaphthone; benzaldehyde; benzoin; the alkyl benzoin ethers; benzophenone; benzoquinone; 1-chloroanthra-quinone; p-diacetyl-benzene; 9,10-dibromoanthracene 9,10-dichloro-anthracene; 4,4-dichlorobenzophenone; thioxanthone; isopropyl-thioxanthone; methylthioxanthone; α,α,α-trichloro-para-t-butyl acetophenone; 4-methoxybenzophenone; 3-chloro-8-nonylxanthone; 3-iodo-7-methoxyxanthone; carbazole; 4-chloro-4′-benzylbenzophenone; fluoroene; fluoroenone; 1,4-naphthylphenylketone; 1,3-pentanedione; 2,2-di-sec.-butoxy acetophenone; dimethoxyphenyl acetophenone; propiophenone; isopropylthioxanthone; chlorothioxanthone; xanthone; maleimides and their derivatives; and mixtures thereof. There are several suitable photoinitiators commercially available from Ciba including Irgacure® 184 (1-hydroxy-cyclohexyl-phenyl-ketone), Irgacure® 819 (bis(2,4,6-trimethylbenzoyl)-phenylphosphineoxide), Irgacure® 1850 (a 50/50 mixture of bis(2,6-dimethoxybenzoyl)-2,4,4-trimethylpentyl-phosphine oxide and 1-hydroxy-cyclohexyl-phenyl-ketone), Irgacure® 1700 (a 25/75 mixture of bis(2,6-dimethoxybenzoyl)=2,4,4-trimethylpentyl-phosphine oxide and 2-hydroxy-2-methyl-1-phenyl-propan-1-one), Irgacure® 907 (2-methyl-1 [4-(methylthio)phenyl]-2-morpholonopropan-1-one), Darocur® MBF (a phenyl glyoxylic acid methyl ester), Irgacure® 2020 Photoinitiator blend (20% by weight of phenylbis(2,3,6-trimethyl benzoyl)phosphine oxide and 80% by weight of 2-hydroxy-2-methyl-1-phenyl-1-propanone) and Darocur® 4265 (a 50/50 mixture of bis(2,4,6-trimethylbenzoyl)-phenylphosphineoxide and 2-hydroxy-2-methyl-1-phenyl-propan-1-one). The foregoing lists are meant to be illustrative only and are not meant to'exclude any suitable photoinitiators. Those skilled in the art will know the concentrations at which photoinitiators are effectively employed and generally the concentration will not exceed about 10% by weight of the radiation-curable coating composition.

Those skilled in the art of photochemistry are fully aware that photoactivators can be used in combination with the aforementioned photoinitiators and that synergistic effects are sometimes achieved when such combinations are used. Photoactivators are well known in the art and require no further description to make known what they are and the concentrations at which they are effective. Nonetheless, one can mention as illustrative of suitable photoactivators, methylamine, tributylamine, methyldiethanolamine, 2-aminoethylethanolamine, allylamine, cyclohexylamine, cyclopentadienylamine, diphenylamine, ditolylamine, trixylylamine, tribenzylamine, n-cyclohexylethyleneimine, piperidine, N-methylpiperazine, 2,2-dimethyl-1,3-bis(3-N-morpholinyl)-propionyloxypropane, and mixtures thereof.

A thermal initiator can be an azo compound, a (hydro)peroxide, or an atom transfer radical polymerization inhibitor such as an alkyl halide, optionally in the presence of accelerators and cationically in the presence of superacids such as the phenyl sulfonium metal salts.

### EXAMPLES

### Example 1

### Synthesis of Salicyl Acrylate

Salicylic acid (141.7 g, Sigma-Aldrich 105910) was dissolved in 1200 mL acetonitrile (Fisher A998) and excess triethylamine (311.4 g, Acros 15791) in a 3 L round-bottom flask continuously purged with nitrogen gas and stirred at room temperature with a large magnetic stir bar. Acryloyl chloride (101 g, Aldrich A24109) dissolved in 200 mL acetonitrile was added dropwise to the salicylic acid solution over 2 h and was stirred overnight at room temperature. The precipitate, primarily triethylamine hydrochloride salt, was removed by vacuum filtration. Hydroquinone (0.040 g, Fluka 53960) was added to inhibit polymerization. Acetonitrile was removed by rotary evaporation, yielding a viscous brown liquid. This crude product was dissolved in methylene chloride (1.5 L), filtered, quickly washed with 1N HCl (1.2 L), and subsequently washed three times with near-saturated aqueous NaCl (300 g/L, 1.2 L per wash). Sufficient NaOH was added to the salt washings to neutralize the pH to 7, to protect against hydrolysis. A yellowish solid crystallized from the methylene chloride, solution. This product was separated by filtration and recrystallized in isopropanol/water (3:1) by dissolving the product at ˜60° C. and cooling the solution at ˜−5° C. overnight. The product was a yellow-tinted solid, revealed by ¹H NMR in CDCl₃ to be salicyl acrylate (δ: 8.13 d, 7.64 t, 7.39 t, 7.20 d, 6.66 d, 6.40 m, 6.05 d, all 1H) with ˜4% free salicylic acid (δ: 7.91 d, 7.51 t, 7.00 d, 6.93 t) as the only apparent impurity.

### Example 2

A. Salicyl acrylate (SAcr) (1.0 g) was dissolved in dimethylformamide (1.5 g) and combined the urethane acrylate material Desmolux VP LS 2308 (urethane acrylate based on hexane diisocyanate trimer and hydroxyalkyl acrylates, dissolved in 20% of hexanediol diacrylate, product of Bayer MaterialScience AG, Leverkusen, Germany) (10 g). Ciba Darocur 4265 (50% 2,4,6-Trimethylbenzoyl-diphenyl-phosphineoxide, 50% 2-Hydroxy-2-methyl-1-phenyl-propan-1-one) was added at 1% of solids as a photoinitiator (0.11 g).

B. Example 2A was repeated, except the salicyl acrylate was, omitted.

### Example 3

A. Example 2A was repeated using Desmolux U 100 (unsaturated aliphatic urethane acrylate, without reactive diluent, hydroxyl content: about 0.3, Bayer MaterialScience AG, Germany)(10 g) in place of Desmolux VP LS 2308.

B. Example 3A was repeated, except the salicyl acrylate was omitted.

### Example 4

A. Example 2A was repeated using Desmolux XP 2513 (unsaturated aliphatic urethane acrylate, without reactive diluent, Bayer MaterialScience AG, Germany) (10 g) in place of Desmolux VP LS 2308.

B. Example 4A was repeated, except the salicyl acrylate was omitted.

### Example 5

A. Example 2A was repeated using Desmolux VP LS 2396 (isocyanate-bearing urethane acrylate, without reactive diluent, hydroxyl content: NCO content approx. 7.5%, Bayer MaterialScience AG, Germany) (10 g) in place of Desmolux VP LS 2308, and with the addition of 0.3% dibutyltin dilaurate by mass (Dabco T-12) (0.037 g) to catalyze moisture curing of the isocyanate functionality of Desmolux VP LS 2396.

B. Example 5A was repeated, except the salicyl acrylate was omitted.

### Example 6

Example 5A was repeated except with 2.0 g salicyl acrylate dissolved in 3.0 g of dimethylformamide.

### Example 7

Example 5A was repeated except with 4.0 g salicyl acrylate dissolved in 6.0 g of dimethylformamide.

### Example 8

### Artificial Urine

An artificial urine recipe was constructed from various literature sources describing the average values of predominant urine chemicals. An aqueous solution was made containing 310 mM urea, 58.4 mM sodium chloride, 39.13 mM potassium chloride, 28 mM ammonium chloride, 2.17 mM calcium chloride, 13.2 mM sodium sulfate, 2.58 mM magnesium sulfate, 8.67 mM sodium dihydrogen phosphate, and 1.71 mM citric acid, and was adjusted to pH 6.06 by addition of NaOH.

### Example 9

### Film-Making and Testing

Each of the above formulations (described in Examples 2 through 7) was drawn onto a glass plate at 8 mil wet thickness (200 μm) and allowed to dry overnight at ambient conditions for removal of dimethylformamide. Solvent-free coatings were passed twice at 20 ft/min under a high-intensity mercury UV bulb (Fusion UV Systems, Inc.); UVA intensity was 1.2 W/cm², UVB 1.0 W/cm², UVC 0.16 W/cm², and visible 0.85 W/cm². All of these coatings were observed to be uniform, transparent, and glossy, and of acceptable mechanical properties.

Films of each sample (0.50 g) were peeled from the glass substrates and immersed in 10 mL of water or artificial urine in sealed vials in ovens at 37° C. or 60° C. At designated time points, samples of the medium were collected.

### Example 10

### Analysis of Salicylic Acid Release

The salicylic acid concentration of each collected sample was measured using a Tecan Safire spectrophotometer to assess the absorbance at 297 nm, using a Hellma quartz-bottomed microtiter 96-well plate. The absorbance intensity of each control (Examples 2B, 3B, 4B, and 5B) were subtracted from the intensity of the corresponding formulation containing salicyl acrylate (Examples 2A, 3A, 4A, and {5A, 6, and 7}, respectively) to control for any signal contributed by the resin or photoinitiator. From the corrected absorbance intensities and using standard calibration curves for water and artificial urine, the concentration of salicylic acid in each sample was calculated. The sensitivity of this technique in measuring salicylic acid was observed to be approximately 1 microgram per milliliter. The salicylic acid release properties of the Example materials are described in Table 1. Release rates varied substantially by formulation. Release of salicylic acid was faster in artificial urine than in water. Release was faster at 60° C. than at 37° C. Formulations that were; otherwise the same but more concentrated in salicyl acrylate released salicylic acid faster on a relative scale. All of the films of this example retained their uniform, transparent, glossy appearance, as well as their acceptable mechanical properties, throughout the experiment.

TABLE 1
Salicylic acid release rates

| | | | Cumulative fraction of |
|---|---|---|---|
| Formulation | Temp. | | salicylic acid released |
| Example | (° C.) | Medium | after 7 days |
| 2A | 60 | water | 0.11 |
| 3A | 60 | water | 0.13 |
| 4A | 60 | water | 0.17 |
| 5A | 60 | water | 0.50 |
| 5A | 37 | water | 0.18 |
| 6 | 60 | water | 0.59 |
| 6 | 60 | art. urine | 0.96 |
| 7 | 60 | water | 0.73 |

### Example 11

Films (0.5 g) made by the methods described in Example 9, using the formulations described in Examples 5A and 5B, were immersed in a 2N aqueous solution of sodium hydroxide. Samples were collected as described in Example 9. Under these conditions, 90% of the observed salicylic acid release occurred within 1 day and the remaining 10% in the second day. The films of this example retained their appearance and mechanical properties after this exposure to 2N sodium hydroxide.

### Example 12

### Pendulum Hardness

Pendulum hardness was measured on samples 5A, 5B, 6, and 7 according to the standard method described in ASTM D4366-95. The results are given in Table 2. Higher salicyl acrylate concentrations resulted in stiffer films.

TABLE 2
Pendulum Hardness

| Formulation | Pendulum Hardness |
|---|---|
| Example | (sec) |
| 5B | 20 ± 1 |
| 5A | 21 ± 1 |
| 6 | 50 ± 7 |
| 7 | 99 ± 7 |

Although the invention has been described in detail in the foregoing for the purpose of illustration, it is to be understood that such detail is solely for that purpose and that variations can be made, therein by those skilled in the art without departing from the spirit and scope of the invention except as it may be limited by the claims.

What is claimed is:

1. A composition comprising the reaction product of:
A) an ethylenically unsaturated compound having at least one group with the following structure or a salt thereof: 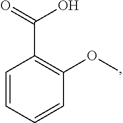
and
B) an ethylenically unsaturated composition having at least one urethane group.

2. A composition according to claim 1, wherein component A) is 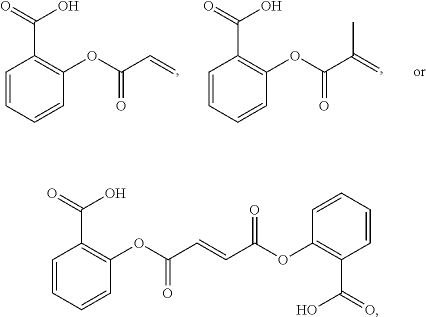
or a salt thereof.

3. A composition according to claim 2, wherein component A) is 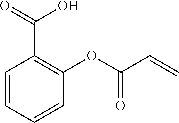
or a salt thereof.

4. A composition according to claim 1, wherein component B) is the reaction product of at least one (i) isocyanate-functional compound and at least one (ii) (meth)acrylate functional compound.

5. A composition according to claim 4, wherein compound B)(i) is selected from the group consisting of hexamethylene diisocyanate, polymers of hexamethylene diisocyanate, isophorone diisocyanate, and polymers of isophorone diisocyanate.

6. A composition according to claim 4, wherein compound B)(ii) is selected from the group consisting of 2-hydroxyethyl acrylate, 2-hydroxyethyl methacrylate, 2-hydroxypropyl acrylate, and 2-hydroxypropyl methacrylate.

7. A polyurethane material which releases salicylic acid by hydrolysis.

8. A method of producing a polyurethane material comprising
1) mixing:
A) an ethylenically unsaturated compound having at least one group with the following structure or a salt thereof: 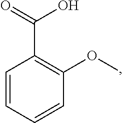
and
B) an ethylenically unsaturated compound having at least one urethane group; and
2) exposing the mixture to actinic radiation.

9. A medical device coated with the polyurethane material produced by the method of claim 8.

10. A composition comprising the reaction product of:
A) an ethylenically unsaturated compound having at least one group with the following structure: 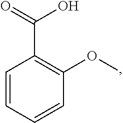
and
B) an ethylenically unsaturated compound,
wherein the composition is capable of reducing the formation of microbial biofilm.

11. A method for modulating biofilm growth on a substrate, comprising coating said substrate with a composition comprising the reaction product of:
A) an ethylenically unsaturated compound having at least one group with the following structure: 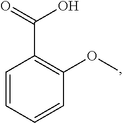
and
B) an ethylenically unsaturated compound.

12. A polymeric composition capable of reducing the formation of microbial biofilmon a substrate, the polymeric composition comprising at least one hydroxyl-containing bioactive agent incorporated in a non-biodegradable polymer backbone, wherein the bioactive agent is released hydrolytically upon exposure to aqueous conditions.

13. A composition according to claim 12, wherein salicylic acid or a salt thereof is the bioactive agent.

14. A composition according to claim 12, wherein the polymeric composition is a polyurethane.

15. A composition according to claim 14, wherein salicylic acid or a salt thereof is the bioactive agent.

16. A composition according to claim 12, wherein the polymeric composition is a (meth)acrylate.

17. A composition according to claim 16, wherein salicylic acid or a salt thereof is the bioactive agent.
